(19) United States
(12) Plant Patent Application Publication
(10) Pub. No.: US 2007/0214529 P1
(43) Pub. Date: Sep. 13, 2007

(54) PELARGONIUM PLANT NAMED 'CLIPS CONSAL'

(76) Inventor: Mitchell E. Hanes, Morgan Hill, CA (US)

Correspondence Address: JONDLE & ASSOCIATES P.C., 858 HAPPY CANYON ROAD SUITE 230, CASTLE ROCK, CO, 80108, US

(21) Appl. No.: 11/369,656

(22) Filed: Mar. 7, 2006

(51) Int. Cl. A01H 5/00

(52) U.S. Cl. PLT/324

(57) ABSTRACT

A pelargonium cultivar particularly distinguished by single-type salmon flowers with red and pale-pink variegation, large inflorescences on strong reddish-brown peduncles well above the foliage, dark green foliage with medium zonation, a medium to tall and upright plant habit, good rain resistance, and a mid-season flowering response in the Spring, is disclosed.

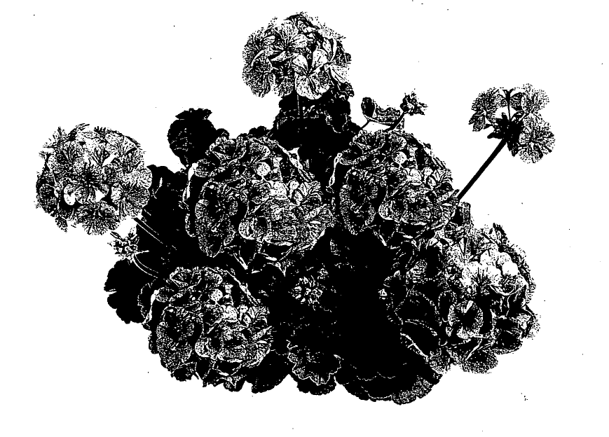

### GENUS AND SPECIES

Pelargonium×hortorum, resp. a hybrid of Pelargonium zonale L'Heritier

### VARIETY DENOMINATION

‘Clips Consal’

### BACKGROUND OF THE NEW PLANT

The present invention comprises a new and distinct cultivar of pelargonium, botanically known as Pelargonium×hortorum, resp. a hybrid of Pelargonium zonale L'Heritier, and hereinafter referred to by the cultivar name ‘Clips Consal’. The new cultivar was a product of a planned breeding program which had the objective of creating new varieties having bi-colored variegated flowers and good outdoor performance. The female parent was the proprietary ‘8831-3’ (unpatented) pelargonium having salmon-red variegated flowers, and the male parent was the proprietary ‘8709-2’ (unpatented) pelargonium, having salmon flowers. The seeds produced by the hybridization in June 2002 and were sown in a greenhouse in September 2002. A single plant selection was chosen for further evaluation and for asexual propagation in the November 2002, in Gilroy, Calif.

The new cultivar was created in 2002 in Gilroy, Calif. and has been asexually reproduced repeatedly by vegetative cuttings and tissue culture in Gilroy, Calif. and Hillscheid, Germany over a three-year period. ‘Clips Consal’ has not been observed under all possible environmental conditions. ‘Clips Consal’ has also been trialed at Gilroy, Calif. and Hillscheid, Germany. ‘Clips Consal’ has been found to retain its distinctive characteristics through successive asexual propagations.

### SUMMARY OF THE INVENTION

The following are the most outstanding and distinguishing characteristics of the new cultivar when grown under normal commercial practices in Hillscheid, Germany.

1. Single-type salmon flowers with red and pale-pink variegated color;

2. Large inflorescences on strong, reddish-brown peduncles well above the foliage;

3. Dark green foliage and leaf zonation of medium distinctness;

4. A medium to tall and upright to spreading plant habit;

5. Good rain resistance; and

6. A mid-season flowering response in the Spring.

### DESCRIPTION OF THE PHOTOGRAPH

This new geranium plant is illustrated by the accompanying photograph which shows the overall plant habit, including inflorescences, buds, and foliage of the plant; the colors shown are as true as can be reasonably obtained by conventional photographic procedures. The photograph is of an un-pinched, 22-week-old plant grown from rooted cuttings in a 5-liter container in the mid-Summer under conditions which approximate those generally used in commercial practice.

### DESCRIPTION OF THE NEW CULTIVAR

The following detailed descriptions set forth the distinctive characteristics of ‘Clips Consal’. The data which defines these characteristics were collected from asexual reproductions carried out in Hillscheid, Germany. The plant history was taken on un-pinched, 13-week-old plants grown in 12-cm pots in a greenhouse in late May 2005. The color readings were taken under natural light in the greenhouse in May 2005. Color references are primarily to the RHS Colour Chart of The Royal Horticultural Society of London (RHS) (2001).

### DETAILED BOTANICAL DESCRIPTION

Classification:
   Botanical.—Pelargonium×hortorum, resp. a hybrid of Pelargonium zonale L'Heritier.
   Common name.—Garden geranium.
Parentage:
   Female parent.—‘8831-3’, a proprietary (unpatented) pelargonium having salmon flowers with red and pink variegation.
   Male parent.—‘8709-2’, a proprietary (unpatented) salmon-flowered pelargonium.
Plant:
   Form.—Shrub; self-branching, pinching not required; initially upright to spreading to later a mounding plant habit.
   Branching habit.—7 branches per plant.
   Height (as measured from the soil surface to the surface of the foliage. canopy, excluding the inflorescence).—20.7 cm for a 13-week-old plant.
   Width (horizontal diameter).—34.7 cm.
   Time to produce a finished flowering plant.—11 weeks for a 12 cm to 14 cm pot in the Spring.
   Outdoor plant performance.—Use in containers, hanging baskets, and as bedding plants.
   Time to initiate and develop roots.—24 days at approximately 20° C.
   Root description.—Fibrous.
Leaves:
   Arrangement.—Alternate.
   Shape.—Kidney-shaped to round with only very weak lobes and with the lowest lobes slightly overlapping.
   Immature leaf.—— Color: — Upper surface: RHS 137C (medium-green) — Lower surface: RHS 137D (medium-green).
   Mature leaf.—— Color: — Upper surface: RHS 137A (dark green) — Lower surface: RHS 137D — Length: 9.4 cm — Width: 5.7 cm.
   Zonation.—— Color: RHS 166A (brown) — Diameter: 7.1 cm, a relatively narrow ring.
   Apex.—Rounded.
   Base.—Cordate.
   Margin.—Bi-crenate and somewhat wavy.
   Texture.—Upper surface is velvety and dull with short pubescence and lower surface has protruding veins in a palmate pattern.
Petioles:
   Length.—7 cm to 10 cm.
   Diameter.—0.4 cm.
   Color.—RHS 137D.
   Texture.—Very short pubescence.
Stems:
   Length.—15 cm to 18 cm.
   Internode length.—1.5 cm to 3.0 cm.
   Diameter (at the midpoint).—1.0 cm to 1.2 cm.
   Color.—Mainly RHS 143B (medium to light green).
   Texture.—Covered by weak pubescence but appears smooth.
Inflorescence buds:
   Length (just before the petals unfold).—1.7 cm.
   Diameter (just before the petals unfold).—0.7 cm.
   Shape.—Narrow and elliptical.
   Sepal color (just before the petals unfold).—RHS 144A (green to yellowish-green) and RHS 178A (brown) at the base.
   Petal color (just before the petals unfold).—RHS 40B (orange-red).
Inflorescence:
   Type.—An umbel composed of about40 to 50 flowers and buds.
   Average number of open inflorescences per plant.—2 to 3 open inflorescences after 13-weeks.
   Number of inflorescences per plant in early September.—15.
   Blooming habit.—Flowers continuously.
   Umbel diameter.—11.4 cm.
   Umbel depth (height).—8.7 cm.
   Umbel shape.—Semi-spherical and tight.
   Lastingness of the umbel on the plant.—16 to 18 days.
   Peduncle.—— Length: 25.5 cm — Diameter: 0.4 cm — Texture: Very fine pubescence — Color: Mainly RHS 183A (brownish).
   Pedicel.—— Length: 3.3 cm. — Diameter: 0.2 cm, with a spur near the base — Texture: Very short pubescence — Color: RHS 174A to RHS 178A (brown).
Corolla:
   Form.—Single-type and nearly radial symmetry.
   Shape.—Nearly round outline with a wide open cup-shape and with some of the petals slightly overlapping.
   Number of petals.—5.
   Petaloids.—None.
   Size.—— Length: 4.9 cm — Width: 4.8 cm — Depth: 1.0 cm.
   Lastingness of the individual flowers on the plant.—5 to 6 days at 18° C.
   Fragrance.—Weak and typical of Pelargonium hortorum.
Petals:
   Upper petals.—— Length: 2.4 cm — Width: 1.7 cm to 1.8 cm — Color: — Upper surface: Variable, partly RHS 52D (pink); RHS 49B (pale-pink) to nearly RHS 49D (white) near the margin; RHS 40C (orange) at the base; RHS 40A (orange-red) irregularly spread streaks and dots. — Lower surface: Partly RHS 52D and partly RHS 49D (nearly white) or RHS 40C (orange) — Markings: RHS 40A (orange-red) irregularly spread streaks and dots.
   Lower petals.—— Length: 2.5 cm — Width: 2.1 cm to 2.2 cm — Color: — Upper surface: Mainly RHS 40C to RHS 40D (orange) — Lower surface: Partly RHS 52D and partly RHS 40C (orange) — Markings: RHS 40A (orange-red) irregularly spread streaks and dots
   Shape.—Obovate for the lower petals to spatulate for the upper petals.
   Apex.—Rounded.
   Base.—Acute.
   Margin.—Entire.
   Texture.—Smooth and silky.
Sepals:
   Number.—5.
   Length.—1.0 cm.
   Width.—0.4 cm for the largest upper sepal and 0.2 cm for the other sepals.
   Shape.—Ligulate or ensiform.
   Apex.—Acute to acuminate.
   Base.—Fused.
   Margin.—Entire.
   Texture.—Weakly pubescent.
   Color.—Mainly RHS 143A (green) with RHS 178A (brown) at the bases for both upper and lower surfaces.
Reproductive organs:
   Androecium.—— Number of anthers: 7 — Filament color: RHS 155A (whitish) near the base to RHS 40C at the top end — Filament length: 0.7 cm — Pollen color: RHS 25A (orange) — Pollen amount: Abundant.
   Gynoecium.—— Number of pistils: 1 — Pistil length: 0.9 cm — Stigma color: RHS 40A (orange-red) — Stigma shape: 5 to 6-lobed at right angles to the style — Style color: RHS 42A — Style length: 0.3 cm — Style shape: Filiform (filament-like).
Fruit/seed set: Develops freely in the late Summer and Fall; comprised of an oval ovary with a long “beak”; the ovary is 0.8 cm in length and 0.5 cm in diameter; the total length of the seed, including the “beak”, is 3.8 cm; when ripe, up to 5 seed grains with spiraled adhesions are set free.
Disease and insect resistance: Average/typical for the species; no special observations made.

### COMPARISON WITH PARENTAL AND COMMERCIAL CULTIVARS

‘Clips Consal’ differs from the female parent ‘8831-3’ (unpatented) in that ‘Clips Consal’ has darker green foliage and a more vigorous growth habit than ‘8831-3’. Additionally, the overall flower color of ‘Clips Consal’ is salmon, while the overall flower color of ‘8831-3’ is salmon-red.

‘Clips Consal’ differs from the male parent ‘8709-2’ (unpatented) in that ‘Clips Consal’ has salmon flowers with red and pale-pink variegation, while ‘8709-2’ has uniform salmon flowers. Additionally, ‘Clips Consal’ has a less vigorous growth habit than ‘8709-2’.

‘Clips Consal’ differs from the commercial variety ‘Amri Conred’ (U.S. Plant Pat. No. 15,880) in that the ground flower color of ‘Clips Consal’ is salmon-orange, while the ground flower color of ‘Amri Conred’ is light red. Additionally, ‘Clips Consal’ has darker green foliage and a more compact plant habit than ‘Amri Conred’.

1. A new and distinct cultivar of pelargonium plant as shown and described herein.
